(19) United States
(12) Patent Application Publication
(10) Pub. No.: US 2006/0166356 A1
(43) Pub. Date: Jul. 27, 2006

(54) Cell culture device

(76) Inventor: Jean-Pierre Ozil, Chaville (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/515,806

(22) PCT Filed: May 16, 2003
(86) PCT No.: PCT/FR03/01499

§ 371 Date: Oct. 28, 2005

(30) Foreign Application Priority Data
May 21, 2002 (FR) 02/06186

(51) Int. Cl. C12M 3/00

(52) U.S. Cl. 435/289.1

(57) ABSTRACT

The invention concerns a device designed in particular for cell culture (10) comprising a chamber (100) wherein are arranged the cells (10), said chamber (100) comprising at least one first conduit (131, 141, 151, 161, 133, 143, 153, 163) for injecting a first type of fluid (M1), at least a second conduit (132, 142, 152, 162, 134, 144, 154, 164) for injecting a second type of fluid (M2). The invention is characterized in that it further comprises a back-injection conduit (181, 182) wheren if one of the ends is laterally connected to the first injection conduit (131, 141, 151, 161, 133, 143, 153, 163) and the other end is connected lo pumping means, the pumping cans being designed, when fluid is injected through the second conduit(s) (132, 142, 152, 162, 134, 144, 154, 164) to draw by suction the fluid contained in the first conduit via the back-1injection conduit (181, 182), such that the fluid (M1) contained in the first conduit is presented from diffusing in the fluid (M2) injected into the chamber (100).

The present invention relates to a cell culture device which allows the treatment thereof with various media while avoiding the handling thereof.

Document FR 2 659 347 (published on Sep. 13, 1991) describes a device for culturing cells comprising a chamber intended to receive the cells. The chamber comprises a horizontal wall formed by two glass plates that are juxtaposed and spaced out so as to form between them a slot having a width that is less than the diameters of the cells. The cells to be treated are placed on the slot. This chamber is intended to contain culture or pulsing media. The various media are injected successively into the chamber via distinct tubes placed above the wall supporting the cells. They are, moreover, evacuated via one or more tube(s) located below the wall.

In that device, the cells are kept on the slot by virtue of the low pressure caused by the suction of the medium by means of the evacuation tube(s).

Such a device is intended in particular for culturing oocytes or fertilized eggs or embryos.

In particular, it can be used for activating experimental oocytes, this activation being necessary for the correct subsequent development of the embryos. To cause this activation, a culture medium is injected into the chamber and the cells to be activated are placed in this culture medium. The ion-rich culture medium is then evacuated and simultaneously replaced with a pulse medium containing Ca²⁺ ions. When the culture medium has been completely evacuated and replaced with the pulse medium, the cells are subjected to a pulse from an electric field, which causes the transient electropermeabilization of their plasma membrane and the penetration of the Ca²⁺ ions into the cells. The pulse medium is then, in turn, evacuated and replaced with the culture medium. These steps are repeated a certain number of times such that the cells are subjected to a controlled series of calcium pulses which triggers their activation.

An advantage of that device is that it makes it possible to treat the cells with various media while avoiding the handling thereof.

However, one problem posed by that type of device is that the replacing of a medium with another is relatively long, which limits the frequency of alternation of the media in the chamber.

For example, when the device is used for activating cells, a minimum time for injecting the pulse medium is necessary (of the order of 40 seconds) in order to replace the culture medium with the pulse medium. This minimum time in fact guarantees sufficient washing of the cells with the pulse medium.

Should this minimum washing time not be observed, the pulse medium would contain residual ions originating from the culture medium. When the electric field was applied, these ions would induce a transmembrane ion current which would cause the cells to be destroyed.

Another problem related to the washing is that the prolonged exposure of the cells in the pulse medium having a low ionic strength disturbs the equilibrium of the cells and exposes them to deleterious effects. In order to preserve cell survival, it is therefore necessary to reduce the cell washing time.

One aim of the invention is to provide a cell culture device which makes it possible to rapidly replace the medium in which the cells are placed.

It has been noted that, when the pulse medium is injected, the ions present in the conduits intended for the injection of the culture medium have a tendency to migrate to the chamber and to contaminate the pulse medium. This phenomenon increases the washing time required in order to obtain a sufficient decrease in the ion concentration in the chamber.

The invention therefore proposes a device intended in particular for cell culture, comprising a chamber in which the cells are placed, said chamber comprising at least one first conduit for injecting a first type of fluid, and at least one second conduit for injecting a second type of fluid, characterized in that it also comprises a back-injection conduit, one of the ends of which is laterally connected to the first injection conduit and the other end of which is connected to pumping means, the pumping means being capable, when fluid is injected through the second conduit(s), of drawing the fluid, contained in the first conduit, by suction via the back-injection conduit, such that the fluid contained in the first conduit is prevented from diffusing into the fluid injected into the chamber.

In this device, the pumping means make it possible to reverse the flow of fluid in the nonactive injection conduit. This transient “back-injection” of fluid opposes the passive diffusion of the ions to the chamber.

This characteristic considerably improves the rate of the replacement of a fluid with another in the chamber and results in replacement times of the order of a second.

According to a preferred embodiment of the invention, the device comprises several conduits for injecting the same type of fluid and mouthpieces of said injection conduits are arranged at regular intervals along a walls of the chamber.

The device may comprise several conduits for injecting each fluid, each conduit for injecting the first type of fluid being superimposed on a conduit for injecting the second type of fluid.

The device may comprise several conduits for injecting each fluid, each conduit for injecting the first type of fluid being placed opposite a conduit for injecting the second type of fluid.

Preferably, the time for replacing one fluid with another in the chamber is of the order of a second. In one embodiment of the invention, the device

comprises electrodes capable of applying to the fluid contained in the chamber a maximum electric field of the order of 10 kV/cm.

The invention also proposes a cell culture method comprising the steps according to which:
the cells are placed in culture in a chamber,
a first type of fluid is injected into the chamber for a given period of time via at least one first injection conduit,
the first type of fluid is evacuated and a second type of fluid is simultaneously injected into the chamber via at least one second injection conduit,
   characterized in that, during the step for injecting the second type of fluid, the fluid contained in the first conduit is drawn by suction, such that the fluid contained in the first conduit is prevented from diffusing into the fluid injected into the chamber.

According to a preferred embodiment of this method, the fluid contained in the first conduit is drawn by suction via a back-injection conduit, one of the ends of which is laterally connected to the first injection conduit and the other end of which is connected to pumping means.

This method may also comprise a step according to which the second type of fluid is evacuated and the first type of fluid is again injected into the chamber via the first injection conduit, and the subsequent steps are repeated such that the cells bathe sequentially in several types of fluid.

According to one embodiment of this method, the cells are subjected to an electric field during the injection of one of the fluids.

Other characteristics and advantages will further emerge from the following description, which is given purely by way of nonlimiting illustration and should be read with regard to the attached figures, among which:

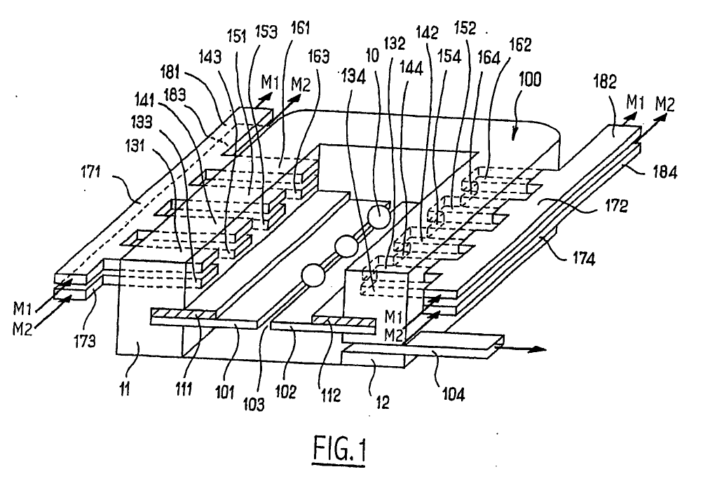

FIG. 1 is a diagram representative of an example of a cell culture device in accordance with an embodiment of the invention,

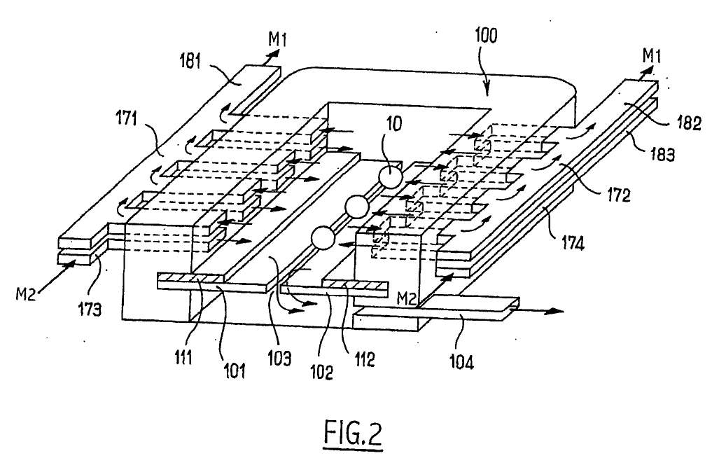

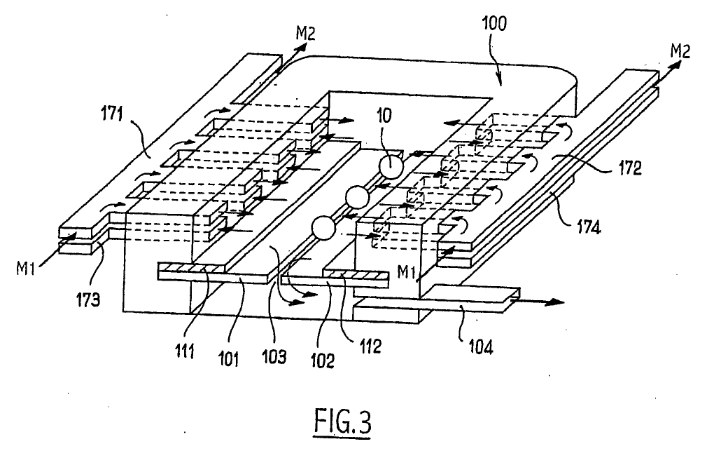

FIGS. 2 and 3 illustrate steps for the functioning of the device in FIG. 1, when the device is used for activating cells,

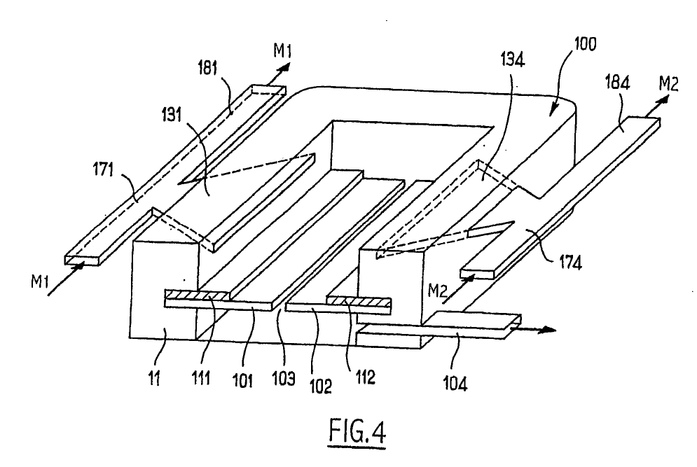

FIG. 4 is a diagram representative of an example of a cell culture device in accordance with another embodiment of the invention,

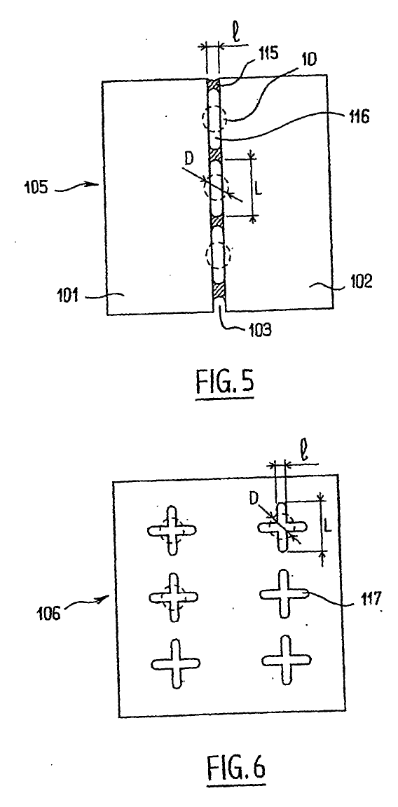

FIG. 5 is a diagram representative of an example of a support element which can be used for supporting the cells to be treated in a chamber,

FIG. 6 is a diagram representative of another example of a support element which can be used for supporting the cells to be treated in a chamber,

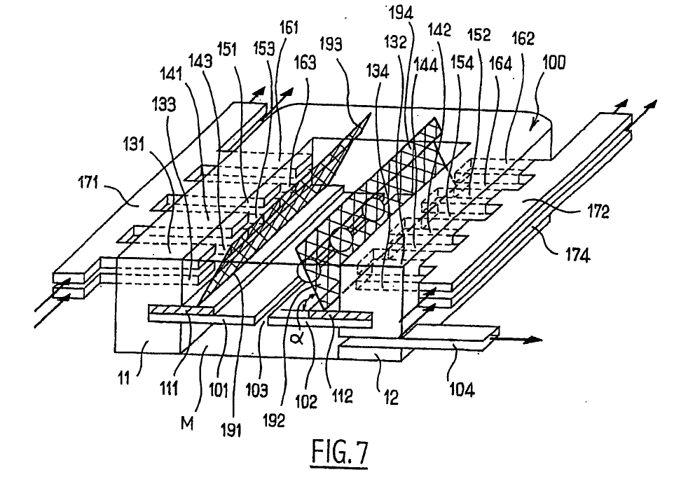

FIG. 7 is a diagram representative of an example of a device for preventing the treatment of the cells being disturbed by gas bubbles.

In FIG. 1, the cell culture device represented comprises a chamber 100 comprising walls delimiting an enclosure intended to contain a fluid medium. This device is, in this example, used for activating oocytes. In the enclosure, a horizontal support element is positioned, formed by the juxtaposition of two glass plates 101 and 102. The glass plates 101 and 102 are held embedded in the side walls 11 and 12 of the chamber. These glass plates 101 and 102 are set apart so as to define, between them, a rectilinear slot 103 that is smaller in width than the diameter of the oocytes 10 to be treated.

The chamber 100 also comprises electrodes 111 and 112 which extend longitudinally on either side of the slot 103.

A culture medium M1, rich in ions, can be brought into the upper part of the enclosure by means of two sets of parallel conduits 131, 141, 151, 161, and 132, 142, 152, 162 opening out, respectively, on the side walls 11 and 12 of the enclosure, placed on either side of the slot 103. The mouthpieces of the conduits 131, 141, 151 and 161 are, respectively, opposite the mouthpieces of the conduits 132, 142, 152 and 162. These mouthpieces are distributed regularly along the walls 11 and 12 of the chamber 100 such that the medium M1 is injected substantially uniformly into the upper part of the enclosure.

A pulse medium M2 containing Ca²⁺ ions can also be brought into the upper part of the enclosure by means of two sets of parallel conduits 133, 143, 153, 163, and 134, 144, 154, 164, similar to the sets of conduits for bringing the medium M1, and superimposed on them.

The medium contained in the chamber and corresponding to the ongoing treatment phase is evacuated by means of an evacuation conduit 104 located at a level lower than that of the support element. The stream of fluid keeps the oocytes 10 stuck to the slot 103, by reduced pressure.

The parallel conduits 131, 141, 151 and 161 are connected to a transverse injection conduit 171. The transverse conduit 171 is connected, at one of its ends, to means for injecting a fluid constituting the medium MI. This conduit 171 extends, at its other end, into a back-injection conduit 181 connected to pumping means that are not represented in this figure.

In a symmetrical manner, the parallel conduits 132, 142, 152 and 162 are connected to a transverse injection conduit 172. The transverse conduit 172 is connected, at one of its ends, to means for injecting medium M1. This conduit 172 extends, at its other end, into a back-injection conduit 182, connected to pumping means.

Similarly, the parallel conduits 133, 143, 153 and 163 are connected, at their end opposite their mouthpiece, to a transverse injection conduit 173. The transverse conduit 173 is connected, at one of its ends, to a reservoir containing a fluid constituting the medium M2. This conduit 173 extends, at its other end, into a back-injection conduit 183 also connected to pumping means.

Finally, in a symmetrical manner, the parallel conduits 134, 144, 154 and 164 are connected, at their end opposite their mouthpieces, to a transverse injection conduit 174. The transverse conduit 174 is connected, at one of its ends, to the reservoir containing the medium M2. This conduit 174 extends, at its other end, into a back-injection conduit 184 connected to pumping means.

The medium contained in the enclosure, corresponding to the ongoing treatment phase, is evacuated by means of a conduit 104 placed at a level lower than that of the oocytes 10. The stream of fluid keeps the oocytes stuck to the slot 103, by reduced pressure.

The steps of an example of treatment which can be carried out by means of this device will now be described.

It is considered that the enclosure is pre-filled with fluid corresponding to the culture medium M1.

In FIG. 2, a first treatment step has been represented, during which the culture medium M1 is replaced with the pulse medium M2. According to this step, as indicated by the arrows, the injection of medium M1 by means of the conduits 171 and 172 is stopped and medium M2 is injected by means of conduits 173 and 174. The medium M1 is evacuated by means of the evacuation conduit 104.

The ions contained in the culture medium M1 which fills the conduits 131, 141, 151, 161, and 132, 142, 152, 162 naturally have a tendency to migrate from the zones with a high ion concentration to the zones with a low ion concentration, i.e. to the fluid contained in the enclosure. In fact, the fluid contained in the enclosure becomes depleted of ions as the medium M1 is replaced with the medium M2, which causes diffusion of the ions from the medium M1 to the medium M2.

In order to counter this phenomenon, the pumping means connected to the conduits 181 and 182 are activated so as to reverse the flow in the injection conduits 131, 141, 151, 161, and 132, 142, 152, 162.

This transient back-injection opposes the diffusion of the ions from the culture medium M1 to the enclosure.

For a nominal flow rate of injection into the enclosure of a nonionic fluid of 100 ml/hour, the ion diffusion can be stopped in less than a second by means of a back-injection flow of 7.9 ml/hour, i.e. slightly less than the nominal flow rate.

The fact that the injection conduits are distributed along the side walls makes it possible to improve the rate of replacement of the medium M1 with the medium M2 at various points of the enclosure.

After this first step of replacement of the medium M1 with the medium M2, the oocytes 10 are completely washed with the pulse medium. They are then subjected to an electric field pulse which can reach 10 kV/cm. The electric field results in the electro-permeabilization of the of the plasma membrane of the oocytes 10 and the penetration of the Ca²⁺ ions into them.

Represented in FIG. 3 is a second treatment step during which the pulse medium M2 is replaced with the culture medium M1. According to this step, as indicated by the arrows, the injection of medium M2 by means of the conduits 173 and 174 is stopped and medium M1 is injected by means of conduits 171 and 172. The medium M2 is evacuated by means of the evacuation conduit 104.

In a manner similar to the first step, the pumping means connected to the conduits 183 and 184 can be activated so as to reverse the flow in the injection conduits 133, 143, 153, 163, and 134, 144, 154, 164. The use of the device in FIG. 1 has been described in the context of the injection of two different types of fluids. It will be understood that this type of device can be applied to the injection of N fluids, it being possible for this injection to be sequential or simultaneous.

The proportional regulating of the flow rates of the injected fluids makes it possible to control, in the enclosure, the temporal variations of simple or complex concentrations of ions, of particles or of molecules.

This device also makes it possible to regulate with precision the frequency of these variations. In the case of the activation of oocytes, the frequency of the calcium pulses can be regulated according to the type of oocytes to be activated.

In FIG. 4, the culture device represented is similar to that of FIGS. 1 to 3, except that it has two injection conduits 131 and 134, these. conduits being intended, respectively, for the injection of the fluids M1 and M2. Each injection conduit 131 and 134 is in the general shape of a triangle, the top of which is connected to a transverse conduit, respectively 171 and 174, and the base of which forms an injection mouthpiece in one of the side walls 11 and 12 of the enclosure.

The mouthpieces of the injection conduits 131 and 134 extend along the walls 11 and 12 of the chamber 100 such that the media M1 and M2 are injected substantially uniformly into the upper part of the enclosure.

This embodiment is particularly advantageous since it makes it possible to generate a virtually laminar uniform injection flow along the walls of the enclosure.

Independently, it has been possible to note that the cells have a tendency to migrate under the effect of the fluid streams generated by the injection and the evacuation of the medium contained in the chamber, it being possible for this migration in particular to result in the agglomeration of these cells.

In the case of the device represented in FIG. 1, the streams of fluid cause the cells to move along the slot of the support element to ward the center of the enclosure where they come together. The cells are compressed against one another. The decrease in space between the cells modifies the efficiency of the washing at the periphery of each cell.

To overcome this drawback, the invention also proposes a device intended in particular for cell culture, comprising a chamber in which the cells are placed, the chamber comprising at least one conduit for injecting fluid and one conduit for drawing fluid by suction, and also a support element comprising orifices on which the cells are placed, characterized in that it comprises a support element comprising orifices intended to receive cells, each orifice being intended to receive a single cell and having at least one minimum dimension that is less than the diameter of the cell and at least one maximum dimension that is greater than the minimum dimension of the orifice, such that the orifice prevents the cell passing through the support element while at the same time allowing the fluid to pass, and the fluid entering the chamber is removed through the orifices, creating a decrease in pressure which ensures that the cells are blocked on the orifices.

The principle of this device can be applied to any type of chamber in which cells must be kept in place and in which a fluid circulates.

In this device, each orifice receives a single cell. This characteristic makes it possible to prevent migration of the cells due to the fluid streams generated by the injection and the evacuation of the medium contained in the chamber.

In addition, these orifices prevent the cells from aggregating with one another, which guarantees good washing. In fact, the washing fluid circulates over the entire periphery of the cell, so as to be evacuated through the orifices. The washing of the cells is efficient, which reduces the washing time required compared with the devices of the prior art.

Preferably, the maximum dimension of an orifice is greater than twice its minimum dimension.

Moreover, the maximum dimension of an orifice is preferably greater than 80 μm.

In addition, the minimum dimension of an orifice is preferably less than 40 μm.

This characteristic makes it possible to prevent part of the cell wall from being hidden in the orifice, which makes it possible to guarantee efficient washing of the cell by the treatment media or the application of an electric field over the entire cell wall.

In one embodiment of the invention, the orifices are oblong in shape.

In another embodiment of the invention, the orifices are in the general shape of a cross.

Typically, the orifices are spaced between 120 and 240 μm apart.

Represented in FIG. 5 is a first type of support element 105. This support element is formed by the juxtaposition of two glass plates 101 and 102 intended to be embedded in the side walls of a chamber, as is represented in FIG. 1. These glass plates 101 and 102 are set apart so as to define with one another a rectilinear slot 103 of width l=20 μm.

The diameter of the cells to be treated varies according to the type of cell. It is considered that these cells generally have a diameter of between approximately 80 μm and 160 μm. The width of the slot is therefore always less than the diameter D of the cells 10 to be treated, whatever their type (these cells are represented as dashed lines).

Polymer joints 115 have been placed at regular intervals in the slot 103 so as to form boxes 116 on which the cells 10 will be placed. The boxes 116 have an oblong shape of length L=2×l=40 μm.

Each box 116 is intended to receive a single cell 10. Since the width l of a box is less than the cell diameter, the cell is held pushed onto the glass plates 101 and 102. Since the length of a box is greater than its width l, the cell is not completely hidden in the box. This characteristic allows the liquid stream to pass on either side of the cell. The washing of the cell therefore remains efficient.

In addition, the fact that the cells 10 are held in separate boxes prevents them from agglomerating.

In a variant, the support element in FIG. 5 is made up of a single glass plate and the boxes 116 are geometrically oblong orifices cut into the glass plate.

The boxes are distributed on the glass plate at regular intervals, preferably of between 120 and 240 μm.

Represented in FIG. 6 is a second type of support element 106. This support element is made up of a single glass plate in which the boxes 117 have been formed. The boxes 117 are orifices in the general shape of a cross. The width l of the arms of the cross is less than the cell diameter D and the length L of each arm of the cross is greater than the width l.

Of course, other forms of boxes can be realized. In order to keep the cell in place on the support element, these boxes have at least one dimension l which is less than the diameter of the cell. In order to guarantee a circulation of fluid around the cells, these boxes have at least one dimension L that is greater than the width l.

However, the dimension l must not be too close to the smallest cell diameter. For example, the dimension l will preferably be less than 40 μm in order to ensure that the cell is kept above the level of the support element. In fact, should the dimension l be too close to the cell diameter, the reduced pressure created by the fluid stream passing through the boxes would cause the cells that are small in diameter to be partially drawn through the boxes by suction. This would result in a portion of the wall of the cells being hidden. This hidden portion of the cell wall would therefore no longer be subjected to the washes with the treatment media or to the application of an electric field. The effectiveness of the treatment applied would as a result be reduced.

Also independently, the formation of gas bubbles on the walls of the hydraulic circuits of the cell culture devices has been noted. These bubbles arise spontaneously in the conduits and are entrained by the fluid stream. They hit against the cells and displace them from their attachment.

In particular, in the case of the culture device in FIG. 1, the cells are entrained and become superimposed between the electrodes. They can create conductivity bridges between the electrodes at the time of the electric pulses. They may sometimes be evacuated with the flow of liquid out of the chamber.

The disturbance of the treatment of the cells by the bubbles means that the treatment is definitively doomed to failure.

To overcome this disadvantage, the invention proposes a device intended in particular for cell culture, comprising a chamber in which the cells are placed, the chamber comprising at least one conduit for injecting fluid and one conduit for drawing fluid by suction, characterized in that it also comprises a grid placed in front of the mouthpieces of the injection conduits.

This grid can be used in any type of chamber in which cells must be kept in place and in which a fluid circulates.

In this device, the grid makes it possible to collect the bubbles at the outlet of the injection conduits.

The bubbles, guided by the grid, then have a tendency to agglomerate at the surface of the fluid of the enclosure. The cells therefore remain in place.

Typically, the grid has a mesh for which the units have dimensions of the order of 100 μm.

Preferably, the grid is placed at a distance of the order of a few tens of millimeters to 1 millimeter from the mouthpieces of the injection conduits.

Also preferably, the grid is sloping at an angle substantially equal to 70 degrees relative to the horizontal.

In one embodiment of the invention, the grid comprises lateral deflectors above the free surface of the fluid so as to prevent bubbles at the surface of the fluid getting round the grid.

Preferably, the grid(s) extend(s) in front of each fluid injection mouthpiece.

Moreover, since the mouthpieces of the conduit are distributed on two opposite side walls of the chamber, the device can comprise two grids, each extending opposite one of the side walls.

Represented in FIG. 7 is a device similar to that in FIG. 1, on which two grids 191 and 192 have been placed symmetrically opposite, respectively, the mouthpieces of the sets of injection conduits 131, 133, 141, 143, 151, 153, 161, 163, and 132, 134, 142, 144, 152, 154, 162, 164, so as to collect the bubbles. The mesh of each grid 191 and 192 has dimensions of the order of 100 μm×100 μm. The grids 191 and 192 having such a mesh cause the bubbles emerging from the injection conduits to deviate, without altering the flow of fluid injected.

The lower part of the grid 191 is placed at a distance of approximately 1 mm from the mouthpieces of the lower conduits 133, 143, 153 and 163. In addition, it is sloping at an angle a of approximately 70 degrees relative to the horizontal.

Similarly, the lower part of the grid 192 is placed at a distance of approximately 1 mm from the mouthpieces of the lower conduits 134, 144, 154 and 164. It is also sloping at an angle a of approximately 70 degrees relative to the horizontal.

The slope of the grids directs the flow of the bubbles to the free surface of the fluid M contained in the chamber.

Some bubbles burst on arriving at the surface of the fluid.

Others, which are more stable, agglomerate. These bubbles can fuse to form larger bubbles.

The surface tensions allow the bubbles to rise above the free surface of the fluid.

The grids 191 and 192 also comprise lateral deflectors 193 and 194 above the free surface of the fluid M. These deflectors prevent the bubbles from following the fluid bridges created by the surface tension forces at the edges of the walls of the chamber 100 in order to rejoin the central part of the enclosure where the cells 10 are placed.

These grids can be made of metal or of plastic.

1. A device intended in particular for cell (10) culture, comprising a chamber (100) intended to receive the cells (10), said chamber (100) comprising at least one first conduit for injecting (131, 141, 151, 161, 133, 143, 153, 163) a first type of fluid (M1), and at least one second conduit for injecting (132, 142, 152, 162, 134, 144, 154, 164) a second type of fluid (M2), characterized in that it also comprises a back-injection conduit (181, 182), one of the ends of which is laterally connected to the first injection conduit (131, 141, 151, 161, 133, 143, 153, 163) and the other end of which is connected to pumping means, the pumping means being capable, when fluid is injected through the second conduit(s) (132, 142, 152, 162, 134, 144, 154, 164), of drawing the fluid, contained in the first conduit, by suction via the back-injection conduit (181, 182), such that fluid (M1) contained in the first conduit is prevented from diffusing into the fluid (M2) injected into the chamber (100).

2. The device as claimed in claim 1, characterized in that it comprises several conduits for injecting the same type of fluid, and in that the mouthpieces of said injection conduits (131, 141, 151, 161, 133, 143, 153, 163; 132, 142, 152, 162, 134, 144, 154, 164) are placed at regular intervals along one of the walls (11, 12) of the chamber (100).

3. The device as claimed in claim 1, characterized in that it comprises an injection conduit (131, 134), the mouthpiece of which extends along one of the walls (11, 12) of the chamber (100).

4. The device as claimed in one of the preceding claims, characterized in that it comprises an injection conduit (131, 134) having a general shape that widens toward the chamber (100).

5. The device as claimed in one of the preceding claims, characterized in that it comprises several injection conduits (131, 141, 151, 161, 133, 143, 153, 163; 132, 142, 152, 162, 134, 144, 154, 164), the mouthpieces of which are placed opposite one another on two opposite walls (11, 12) of the chamber (100).

6. The device as claimed in one of the preceding claims, characterized in that the replacing of one fluid with another in the chamber is of the order of a second.

7. The device as claimed in one of the preceding claims, characterized in that it comprises electrodes (111, 112) capable of applying to the fluid contained in the chamber a maximum electric field of the order of 10 kV/cm.

8. The device as claimed in one of the preceding claims, characterized in that it comprises a support element (105, 106) comprising orifices (116, 117) intended to receive cells (10), characterized in that each orifice (116, 117) is intended to receive a single cell (10) and has at least one minimum dimension (l) that is less than the diameter (D) of the cell (10) and at least one maximum dimension (L) that is greater than the minimum dimension (l) of the orifice, such that the orifice (116, 117) prevents the cell (10) passing through the support element (105, 106) while at the same time allowing the fluid to pass, and the fluid entering the chamber (100) is removed through the orifices (116, 117), creating a decrease in pressure which ensures that the cells (10) are blocked on the orifices.

9. The device as claimed in claim 8, characterized in that the maximum dimension (L) of an orifice (116, 117) is greater than twice its minimum dimension (l).

10. The device as claimed in either of claims 8 and 9, characterized in that the maximum dimension (L) of an orifice is greater than 80 μm.

11. The device as claimed in one of claims 8 to 10, characterized in that the minimum dimension (l) of an orifice is less than 40 μm.

12. The device as claimed in one of claims 8 to 11, characterized in that the orifices (116, 117) are oblong in shape.

13. The device as claimed in one of claims 8 to 11, characterized in that the orifices (116, 117) are in the general shape of a cross.

14. The device as claimed in one of claims 8 to 13, characterized in that the orifices are spaced between 120 and 240 μm apart.

15. The device as claimed in one of the preceding claims, characterized in that it comprises a grid (191, 192) placed in front of the mouthpieces of the injection conduits (131, 141, 151, 161, 133, 143, 153, 163; 132, 142, 152, 162, 134, 144, 154, 164).

16. The device as claimed in claim 15, characterized in that the grid has a mesh, the units of which have dimensions of the order of 100 μm.

17. The device as claimed in either of claims 15 and 16, characterized in that the grid (191, 192) is placed at a distance of between 0.5 and 1 mm from the mouthpieces of the injection conduits (131, 141, 151, 161, 133, 143, 153, 163; 132, 142, 152, 162, 134, 144, 154, 164).

18. The device as claimed in one of claims 15 to 17, characterized in that the grid (191, 192) is sloping at an angle substantially equal to 70 degrees relative to the horizontal.

19. The device as claimed in one of claims 15 to 18, characterized in that the grid (191, 192) comprises lateral deflectors (193, 194) above the free surface of the fluid in order to prevent bubbles at the surface of the fluid M getting round the grid (191, 192).

20. The device as claimed in one of claims 15 to 19, characterized in that the grid(s) extend(s) in front of each fluid injection mouthpiece (131, 141, 151, 161, 133, 143, 153, 163; 132, 142, 152, 162, 134, 144, 154, 164).

21. The device as claimed in claim 20, characterized in that, since the mouthpieces of the conduits (131, 141, 151, 161, 133, 143, 153, 163; 132, 142, 152, 162, 134, 144, 154, 164) are distributed on two opposite side walls (11, 12) of the chamber (100), it comprises two grids (191, 192), each extending opposite one of the side walls (11, 12).

22. The device as claimed in one of claims 15 to 21, characterized in that the grid(s) is(are) made of metal or of plastic.

23. A cell (10) culture method, according to which:
the cells (10) are placed in culture in a chamber (100),
a first type of fluid (M1) is injected into the chamber (100) for a given period of time via at least one first injection conduit (131, 141, 151, 161, 132, 142, 152, 162),
the first type of fluid (M1) is evacuated and a second type of fluid (M2) is simultaneously injected into the chamber (100) via at least one second injection conduit (133, 143, 153, 163, 134, 144, 154, 164),
characterized in that, during the step for injecting the second type of fluid (M2), the fluid (M1) contained in the first conduit (131, 141, 151, 161, 132, 142, 152, 162) is drawn by suction, such that fluid (M1) contained in the first conduit (131, 141, 151, 161, 132, 142, 152, 162) is prevented from diffusing into the fluid (M2) injected into the chamber (100).

24. The method as claimed in claim 23, characterized in that the fluid (M1) contained in the first conduit is drawn by suction via a back-injection conduit (181, 182), one of the ends of which is laterally connected to the first injection conduit (131, 141, 151, 161, 133, 143, 153, 163) and the other end of which is connected to pumping means.

25. The method as claimed in either of claims 23 and 24, characterized in that it also comprises a step according to which the second type of fluid (M2) is evacuated and the first type of fluid (M1) is again injected into the chamber (100) via the first injection conduit (131, 141, 151, 161, 132, 142, 152, 162), and in that the subsequent steps are repeated such that the cells (10) bathe sequentially in several types of fluid.

26. The method as claimed in one of claims 23 to 25, characterized in that, during the injection of one of the fluids, the cells (10) are subjected to an electric field.

27. The method as claimed in one of the preceding claims, characterized in that the step for replacing one fluid with another in the chamber lasts approximately 1 second.
